(19) United States
(12) Patent Application Publication
(10) Pub. No.: US 2022/0346838 A1
(43) Pub. Date: Nov. 3, 2022

(54) Blockage Removal

(71) Applicant: Mark Anthony Cutino, Bremerton, WA (US)

(72) Inventor: Mark Anthony Cutino, Bremerton, WA (US)

(21) Appl. No.: 17/866,419

(22) Filed: Jul. 15, 2022

Related U.S. Application Data
(63) Continuation-in-part of application No. 15/818,247, filed on Nov. 20, 2017, now Pat. No. 11,419,622.

(51) Int. Cl. A61B 17/50; A61M 16/04; A61B 17/24; A61M 1/00; A61B 1/267; A61B 17/00

(52) U.S. Cl. CPC A61B 17/50; A61M 16/049; A61B 17/24; A61M 1/802; A61B 1/267; A61B 2017/00734

(57) ABSTRACT

Techniques for blockage removal are provided, which may provide immediate and effective aid to a choking victim. A blockage removal device may comprise a mouthpiece disposed on an inlet. The inlet may be coupled to a chamber configured to hold a vacuum generated by a vacuum pump. A vacuum gauge may display information to a user, who may open or close a valve to provide or remove a vacuum to attempt to remove a blockage.

### FIELD

This disclosure relates generally to blockage removal.

### BACKGROUND

Choking is a common occurrence that may become life-threatening if not resolved quickly. Choking may entail the mechanical obstruction of airflow from the environment into the lungs. Choking may occur when an airway becomes constricted or obstructed, for example, when a foreign object lodges in a throat or windpipe.

Choking may be partial or complete. Partial choking may allow some, but insufficient, airflow into the lungs, whereas complete choking may cut off airflow entirely. Because choking deprives a brain of oxygen, immediate aid is crucial. Prolonged or complete choking may result in asphyxia, which may lead to anoxia or death. Conventional methods to aid a choking victim, for example, abdominal thrusts, chest thrusts, cardiopulmonary resuscitation (CPR), or using a finger to dislodge an obstruction are often inadequate and insufficient in adapting to a rescuer's or victim's particular physical conditions or limitations.

### SUMMARY

The following presents a simplified summary of the disclosure to provide a basic understanding to the reader. This summary is not an extensive overview of the disclosure, nor does it identify key or critical elements of the claimed subject matter or define its scope. Its sole purpose is to present some concepts disclosed in a simplified form as a precursor to the more detailed description that is later presented.

The instant application discloses, among other things, blockage removal, which may provide ways to remove a blockage from a passageway. For example, if a person is choking, this device may help dislodge an item from an airway. In one implementation, blockage removal may comprise a mouthpiece coupled to a chamber configured to hold vacuum generated by a vacuum pump. A pressure transducer may sense an amount of vacuum, for example, and display information on a pressure gauge. A user may control whether the vacuum is applied to the choking person by a button valve, which may be used to provide or release the vacuum to remove the blockage. The blockage removal process may be repeated until a victim's airway has been cleared or choking has been resolved.

Many of the attendant features may be more readily appreciated as they become better understood by reference to the following detailed description considered in connection with the attached drawings.

### BRIEF DESCRIPTION OF THE DRAWINGS

FIG. 1 illustrates a diagrammatic top view of a Blockage Removal device, according to one implementation.

FIG. 2 illustrates a diagrammatic perspective view of a Blockage Removal device utilized on a choking victim, according to one implementation.

FIG. 3 illustrates a flow diagram of a Blockage Removal process, according to one implementation.

Like reference numerals are used to designate like parts in the accompanying drawings.

### DETAILED DESCRIPTION

A more particular description of certain implementations of Multi-Author Document Collaboration may be had by references to the implementations shown in the drawings that form a part of this specification, in which like numerals represent like objects.

The illustrated operations in the description show certain events occurring in a certain order. One skilled in the art will recognize that certain operations may be performed in a different order, modified, or removed. Moreover, steps may be added to the described logic and still conform to the described implementations.

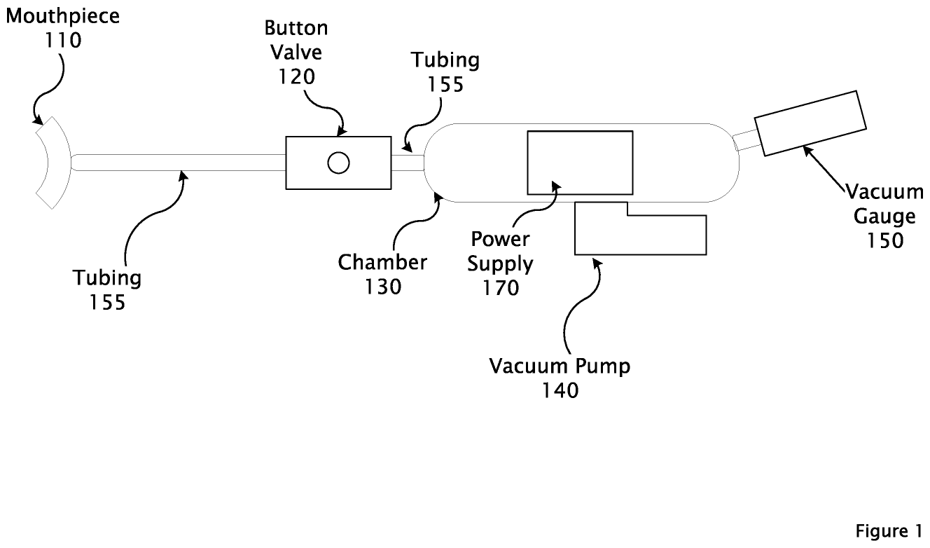

FIG. 1 illustrates a diagrammatic top view of a Blockage Removal 100 device, according to one embodiment. In one implementation, it may comprise an inlet upon which Mouthpiece 110 is disposed and coupled to Chamber 130 by Tubing 155. Chamber 130 may comprise an evacuated cylinder configured to hold vacuum generated by Vacuum Pump 140. Vacuum Gauge 150 may display an amount of Vacuum in Chamber 130. Button Valve 120 may allow a user to expose or block the blockage to the vacuum.

A person skilled in the art will understand that Blockage Removal 100 or its components may comprise various shapes, colors, or sizes, and may be made of any material, for example, polymers, composites, wood, rubber, metal, or other materials.

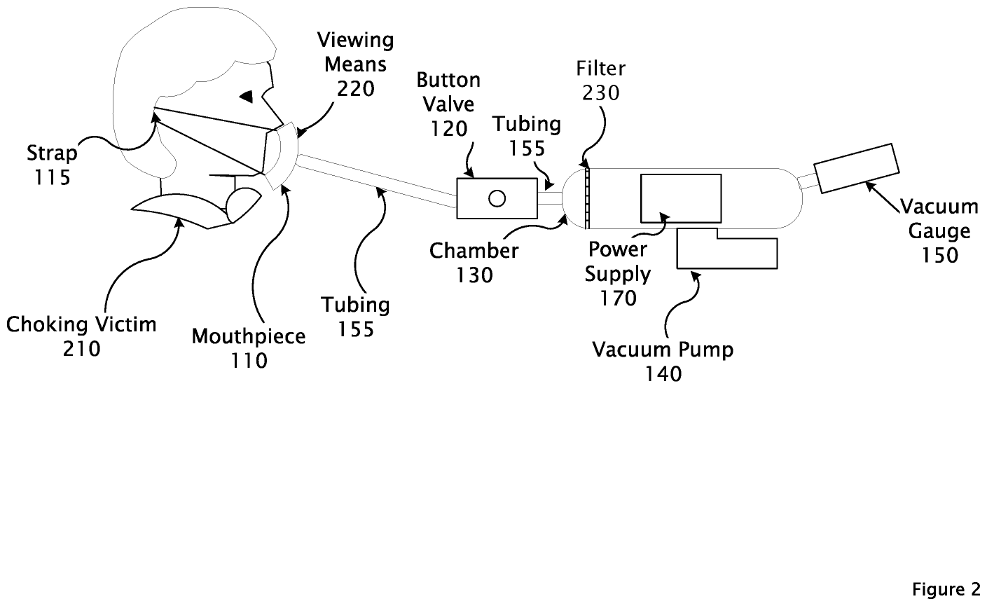

FIG. 2 illustrates a diagrammatic perspective view of a Blockage Removal 100 device utilized on a choking victim, according to one embodiment. Blockage Removal 100 may be operable to provide immediate and effective aid to Choking Victim 210. Choking Victim 210 may comprise a person or animal whose airway is constricted or obstructed, for example, because a foreign object has entered a throat or windpipe. Choking Victim 210 may also include any non-living thing that otherwise experiences a passageway blockage. In one implementation, a user may place Mouthpiece 110 over an opening, for example, a mouth, of Choking Victim 210. Mouthpiece 110 may create an airtight or watertight seal around the mouth of Choking Victim 210. In another implementation, a Strap 115 or other attachment means may facilitate securing Mouthpiece 110, Blockage Removal 100, or any of its components to Choking Victim 210.

Mouthpiece 110 may be disposed on an inlet of Blockage Removal 100. Mouthpiece 110 may couple to Chamber 130 by Tubing 155, which may be disposed between Mouthpiece 110 and Chamber 130. Tubing 155 may comprise a clear or translucent material that may allow a user to see and determine whether an obstruction has been removed from an airway of Choking Victim 210, for example. Tubing 155 may be disposed at any angle.

Chamber 130 may comprise an evacuated cylinder configured to hold a vacuum generated by Vacuum Pump 140. Vacuum Pump 140 may contain an impeller that uses centrifugal force to generate the vacuum in one implementation. In another embodiment, Vacuum Pump 140 may be operable to generate a positive displacement of volume, for example.

Vacuum Gauge 150 may comprise a pressure transducer operable to sense an amount of vacuum inside Chamber 130. Vacuum Gauge 150 may display an amount of Vacuum in Chamber 130.

Instead of utilizing a constant stream of negative pressure found in conventional vacuum devices, Blockage Removal 100 may achieve an instantaneous jolt or change in pressure conducive to quickly and safely dislodging a foreign object from an airway of Choking Victim 210, or otherwise resolving a choking incident, without causing further discomfort, damage, or injury.

Vacuum Pump 140 may couple to Power Supply 170, which may be, for example, a battery pack, a connection to a power outlet, or solar power.

In another implementation, Blockage Removal 100 may include Viewing Means 220, for example, a translucent material of Mouthpiece 110, a light, magnifier, camera, or display screen to see inside a blocked passageway, for example, an obstructed airway, while using a Blockage Removal 100 device.

In yet another implementation, Blockage Removal 100 may provide a means for cleaning, filtering, or removing waste or objections from Blocking Removal 100 device or its components; for example, it may include a screen or Filter 230. Blockage Removal 100 may provide visual or audio prompts or instructions in yet another implementation.

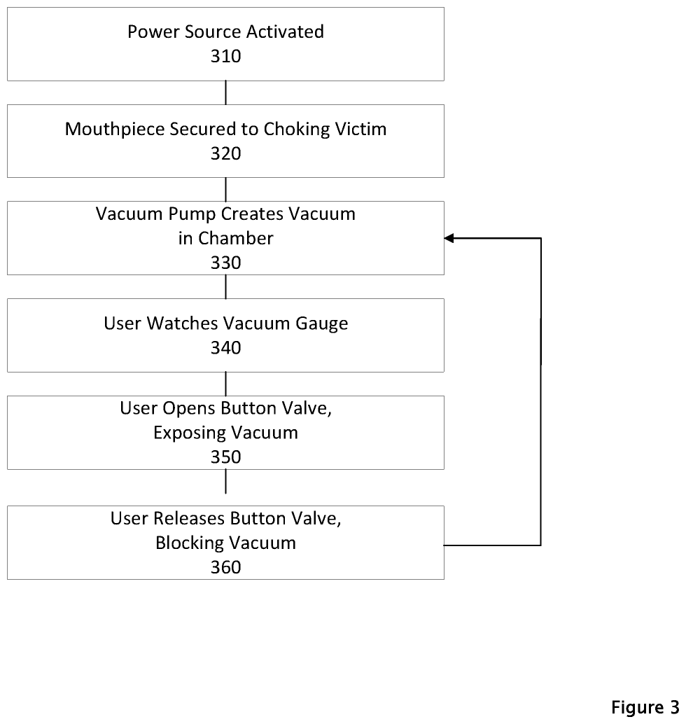

FIG. 3 illustrates a flow diagram of a process utilizing a Blockage Removal process, according to one embodiment.

At Power Source Activated 310, a user may press a button, move a switch, send a voice-activated command, or execute another means to enable power to a Blockage Removal device. The user may be a rescuer, bystander, or choking victim using the device on one's self, for example. The power source may comprise electrical, solar, or another type of power.

At Mouthpiece Secured to Choking Victim 320, an inlet equipped with a mouthpiece may be placed over an opening, for example, a mouth of the choking victim. The mouthpiece may create a fluid-tight seal to prevent interference with the vacuum or to achieve complete or proper suction.

At Vacuum Pump Creates Vacuum in Chamber 330, the vacuum pump may utilize an impeller that uses centrifugal force to generate vacuum in the chamber. In another embodiment, the vacuum pump may generate a positive displacement of volume, for example.

At User Watches Vacuum Gauge 340, the user may watch for an amount of vacuum in the chamber, for example, an amount in units of millimeters of mercury (mmHg).

At User Opens Button Valve, Exposing Vacuum 350, the vacuum may allow Blockage Removal to achieve an instantaneous jolt or change in pressure conducive to quickly and safely removing an obstruction from a passageway, for example, dislodging a foreign object from an airway of a choking victim, or otherwise resolving a choking incident without causing further discomfort, damage, or injury.

At User Releases Button Valve, Blocking Vacuum 360, the vacuum may be at least temporarily blocked. The user may repeat at Vacuum Pump Creates Vacuum in Chamber 330. For example, the user may repeat the cycle at 10-second intervals or any other duration, or until full vacuum has been achieved, a victim's airway has been cleared, an obstruction has been removed, or choking has been resolved.

1. A device, comprising:
an inlet,
a mouthpiece disposed on the inlet;
a chamber, the chamber operable to hold a vacuum, wherein the vacuum comprises air pressure lower than atmospheric pressure;
a vacuum pump, the vacuum pump operable to create the vacuum in the chamber;
a vacuum gauge, the vacuum gauge operable to measure and display the vacuum inside the chamber;
a valve, the valve operable to open or close, wherein opening or closing of the valve exposes or blocks the vacuum; and
a power source.

2. The device of claim 1, wherein the mouthpiece includes a fluid-tight seal.

3. The device of claim 1, wherein the mouthpiece includes a strap.

4. The device of claim 1, wherein the mouthpiece comprises an anti-microbial material.

5. The power source of claim 1, further comprising items from the list containing a battery pack, a solar cell, wall plug, an alternating current (AC) to direct current (DC) converter, and a direct current (DC) to an alternating current (AC) converter.

6. The device of claim 1, wherein the device comprises any combination of materials from the list containing polymers, composites, wood, rubber, or metal.

7. The device of claim 1, further comprising a filter.

8. The device of claim 1, further comprising a viewing means from the list containing a translucent material of the mouthpiece, a light, a magnifier, a camera, or a display screen.

9. A blockage removal method, comprising:
activating a power source;
securing a mouthpiece disposed on an inlet to a choking victim;
enabling a vacuum pump to create vacuum in a chamber;
enabling a vacuum gauge to display vacuum pressure;
opening a valve to expose the vacuum;
closing the valve to block the vacuum at least temporarily; and
repeating the blockage removal method to restart.
